(19) United States
(12) Patent Application Publication
(10) Pub. No.: US 2020/0117195 A1
(43) Pub. Date: Apr. 16, 2020

(54) MEDICAL NETWORK SYSTEM AND EXTERNAL DEVICE

(71) Applicant: HONDA MOTOR CO., LTD., Minato-ku, Tokyo (JP)

(72) Inventors: Yuji Yasui, Wako-shi (JP); Hisao Asaumi, Wako-shi (JP); Shion Tokunaga, Wako-shi (JP); Masashi Yuki, Wako-shi (JP); Yo Ito, Tokyo (JP); Hirotaka Uchitomi, Tokyo (JP)

(21) Appl. No.: 16/621,260

(22) PCT Filed: Jun. 12, 2018
(86) PCT No.: PCT/JP2018/022424

§ 371 Date: Dec. 11, 2019

(30) Foreign Application Priority Data
Jun. 16, 2017 (JP) 2017-118692

(51) Int. Cl. G05D 1/00; G16H 40/20; G16H 40/40; G16H 50/20

(52) U.S. Cl. CPC G05D 1/0088; G16H 40/20; G05D 2201/0213; G16H 50/20; G05D 2201/0206; G16H 40/40

(57) ABSTRACT

A medical network system includes medical examination equipment 300 and a medical diagnosis terminal communicator 420 installed at a community center CC, an automated driving vehicle 200 in which a round doctor DR rides, and a determiner 110 that determines a next vehicle allocation schedule of the automated driving vehicle 200. The automated driving vehicle 200 includes a communication device and a navigation device. The medical diagnosis terminal communicator 420 transmits a medical examination result according to the medical examination equipment 300 to the communication device disposed in the automated driving vehicle 200. The navigation device outputs biological information based on the medical examination result received by the communication device to the round doctor DR.

### TECHNICAL FIELD

The present invention relates to a medical network system and an external device.

Priority is claimed on Japanese Patent Application No. 2017-118692, filed Jun. 16, 2017, the content of which is incorporated herein by reference.

### BACKGROUND ART

In regions having a small population and the like, establishing a hospital or the like may be difficult, and doctors may need to make house calls by visiting the houses of patients and the like. As a technology for improving the convenience of a house call patient and other medical related persons when a doctor makes a house call, conventionally, there is a home medical care support system that notifies a user terminal disposed at a next house call location at a doctor's scheduled house call time (for example, see Patent Document 1).

### CITATION LIST

### Patent Document

### [Patent Document 1]

Japanese Unexamined Patent Application, First Publication No. 2014-229245

### SUMMARY OF INVENTION

### Technical Problem

However, in the home medical care support system disclosed in Patent Document 1 described above, although a user is notified of a doctor's scheduled house call time, it is difficult to regulate the length of stay of a doctor at a user's home. For this reason, when a doctor visits user's homes in a specific region or the like, the time spent at a user's home may become long, and the round may not be performed efficiently.

Thus, an object of the present invention is to provide a medical network system and an external device enabling a doctor to make a round efficiently inside a specific region.

### Solution to Problem

(1): A medical network system including: a medical diagnosis terminal that is installed at a facility at which medical examination equipment is disposed and includes a first communicator; an automated driving vehicle in which a doctor rides and includes a second communicator and an information output; and a determiner that determines a vehicle allocation schedule of the automated driving vehicle such that it travels inside a specific region, in which the first communicator directly or indirectly transmits a medical examination result according to the medical examination equipment to the second communicator, and the information output outputs information based on the medical examination result received by the second communicator to the doctor.

(2): The medical network system according to (1) further including a third communicator that is disposed in parallel with the determiner, in which the first communicator transmits the medical examination result according to the medical examination equipment to the third communicator, the determiner changes the vehicle allocation schedule on the basis of the medical examination result received by the third communicator, and the third communicator transmits the received medical examination result to the second communicator together with the changed vehicle allocation schedule.

(3): An external device including: a medical diagnosis terminal that is installed at a facility at which medical examination equipment is disposed; an external terminal that includes a determiner determining a vehicle allocation schedule of an automated driving vehicle in which a doctor rides such that the automated driving vehicle travels inside a specific region; and a doctor terminal that is operated by the doctor, in which the external terminal transmits the vehicle allocation schedule determined by the determiner to the doctor terminal.

(4): The external device according to (3), in which the doctor terminal relates to a scheduling of a vehicle according to the received vehicle allocation schedule.

(5): In the external device according to (3) or (4), the external terminal transmits authentication information of the automated driving vehicle to the doctor terminal.

(6): In the external device according to any one of (3) to (5), the determiner estimates an expected arrival time of the automated driving vehicle at a predetermined location, and the expected arrival time that is estimated is transmitted to the doctor terminal.

(7): In the external device according to any one of (3) to (6), the doctor terminal transmits treatment-related information based on a result of a primary diagnosis executed by the doctor to the external terminal, and the determiner changes the vehicle allocation schedule on the basis of the treatment-related information transmitted from the doctor terminal.

(8): In the external device according to (7), the determiner generates necessary equipment-related information on the basis of the treatment-related information, and the necessary equipment-related information is transmitted to at least one of the automated driving vehicle and the medical diagnosis terminal.

### Advantageous Effects of Invention

According to (1), the first communicator transmits a medical examination result to the second communicator, and the information output outputs the transmitted medical examination result to the doctor. For this reason, the doctor riding in the automated driving vehicle can perform a primary diagnosis of users in the automated driving vehicle when moving to a user house. As a result, a primary diagnosis time at the user house can be eliminated, and thus a staying time at the user house can be shortened. As a result, the doctor can efficiently round inside a specific region.

According to (2), the determiner changes the vehicle allocation schedule on the basis of a medical examination result transmitted by the first communicator and transmits the changed vehicle allocation schedule to the second communicator. For this reason, in a case in which there is a user with emergency needs or the like due to a result of a medical examination according to medical examination equipment, the determiner can adjust the vehicle allocation schedule. Accordingly, a doctor can efficiently round inside a specific region while handling an emergency of a user.

According to (3), the external terminal transmits the vehicle allocation schedule determined by the determiner to the doctor terminal. For this reason, the doctor can efficiently perform a medical examination and round.

According to (4), the doctor terminal transmits riding information relating to a scheduling of a vehicle according to the received vehicle allocation schedule to the determiner. For this reason, it is possible to reduce the effort when scheduling of a vehicle according to the vehicle allocation schedule in the determiner.

According to (5), authentication information of the automated driving vehicle is transmitted to the doctor terminal. For this reason, erroneous recognition of the automated driving vehicle in which a doctor rides can be prevented.

According to (6), an expected arrival time of the automated driving vehicle at a predetermined location that is estimated by the determiner is transmitted to the doctor terminal. For this reason, a schedule during the time of a doctor's rounding can be easily understood.

According to (7), the determiner changes the vehicle allocation schedule on the basis of the treatment-related information transmitted from the doctor terminal. For this reason, as a result of the primary diagnosis, the vehicle allocation schedule according to the diagnosis result of the user can be generated.

According to (8), the necessary equipment-related information is transmitted to at least one of the automated driving vehicle and the medical diagnosis terminal. For this reason, necessary tools and the like can be provided at the destination of the doctor.

### BRIEF DESCRIPTION OF DRAWINGS

FIG. 1 is a configuration diagram of a medical network system.

FIG. 2 is a configuration diagram of an automated driving vehicle.

FIG. 3 is an explanatory diagram showing map information of a round region.

FIG. 4 is an explanatory diagram showing user information of users in a round region.

FIG. 5 is an explanatory diagram showing a vehicle allocation schedule of a round doctor.

FIG. 6 is an explanatory diagram showing biological information of a user.

FIG. 7 is an explanatory diagram showing biological information of a user according to a second embodiment.

FIG. 8 is a flowchart showing the sequence of a vehicle allocation schedule generating process according to the second embodiment.

FIG. 9 is an explanatory diagram showing vehicle allocation schedules of a round doctor before and after change.

FIG. 10 is a flowchart showing the sequence of a round route generating process according to the second embodiment.

FIG. 11 is a configuration diagram of a medical network system according to a third embodiment.

FIG. 12 is a sequence diagram showing a process executed by a medical network system according to the third embodiment.

### DESCRIPTION OF EMBODIMENTS

Hereinafter, a medical network system according to the present invention will be described with reference to the drawings.

The medical network system is a system that supports a doctor's round of user houses by causing an automated driving vehicle, in which the doctor rides, to travel inside a specific region in which there are a plurality of users requiring medical examination by a doctor (hereinafter, referred to as a “round region”). In the medical network system, biological information of users is acquired using medical examination equipment installed in a facility disposed in a round region or the like, and a doctor executes primary diagnosis by referring to the biological information while riding in an automated driving vehicle. Subsequently, the doctor makes a house call to a user's home when rounding the region and executes secondary diagnosis face-to-face with a user.

### First Embodiment

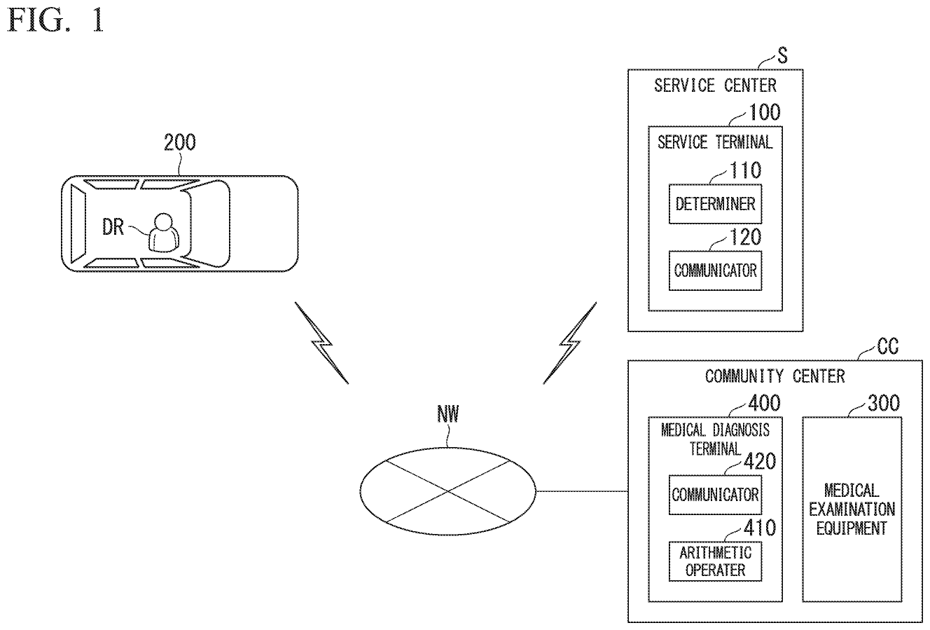

FIG. 1 is a configuration diagram of a medical network system. As shown in FIG. 1, the medical network system, for example, includes a service terminal 100, an automated driving vehicle 200, medical examination equipment 300, and a medical examination terminal 400. For example, the service terminal 100 is disposed in a service center S. For example, the medical examination equipment 300 and the medical examination terminal 400 are installed at a community center CC that is a facility built inside a round region.

The service terminal 100 includes a determiner 110 and a service terminal communicator (one example of a third communicator) 120. For example, the determiner 110 of the service terminal 100 includes a processor such as a CPU or the like and various storage devices. Map information of a round region, user information, and the like are stored in the various storage devices of the determiner 110. The service terminal communicator 120 is disposed in parallel with the determiner 110 and transmits/receives information between the determiner 110 and the automated driving vehicle 200 and the medical examination terminal 400.

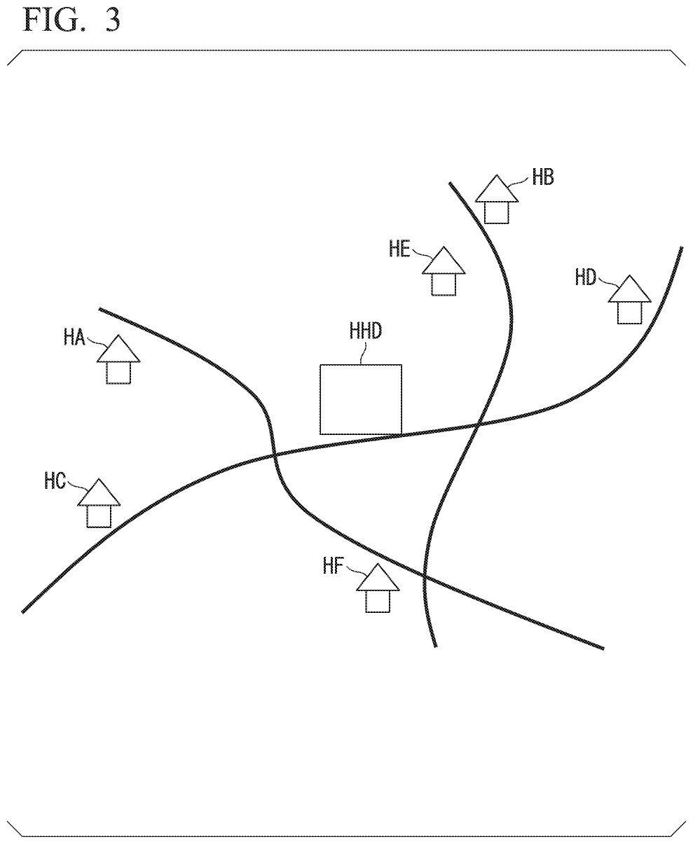

As shown in FIG. 3, a map of the round region and locations of user houses on the map are included in the map information of the round region. An A house HA, a B house HB, a C house HC, a D house HD, an E house HE, and an F house HF are included in the user houses. In addition, a location of a doctor house HHD that is a house of a doctor is also included in the map information.

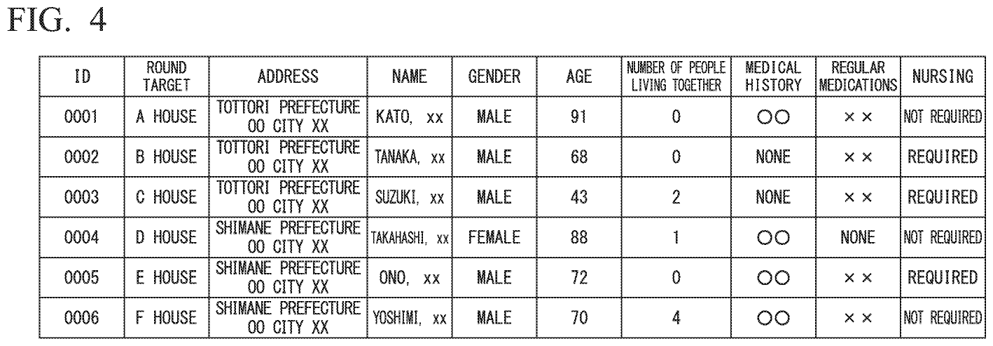

As shown in FIG. 4, information of an ID number of a user, an address, a name, a gender, an age, the number of people living with the patient, an anamnesis, regular medications, and necessity/non-necessity of nursing is included in the user information. As such user information, user information of each of users who are residents of the A house HA, the B house HB, the C house HC, the D house HD, the E house HE, and the F house HF is stored.

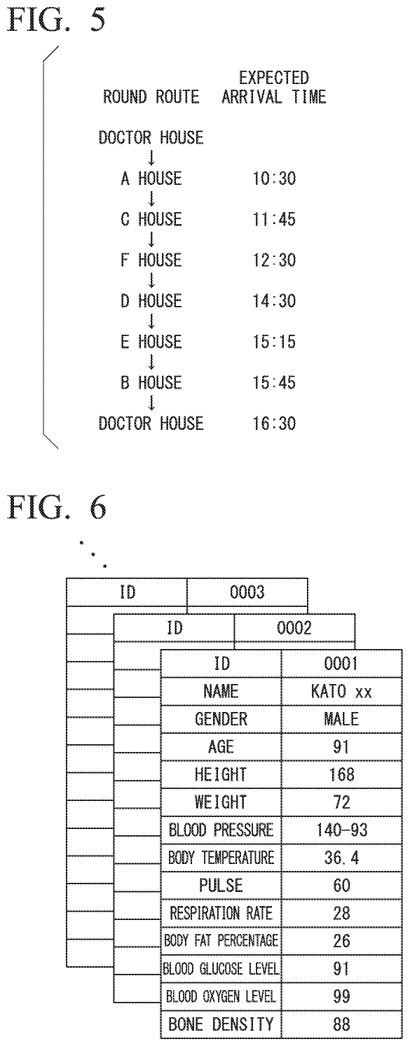

The determiner 110 determines a vehicle allocation schedule including destinations around which the automated driving vehicle 200 in which the doctor is riding rounds, a round order of the destinations, and expected arrival times at the destinations on the basis of the map information shown in FIG. 3 and the user information shown in FIG. 4. The vehicle allocation schedule may be determined using an appropriate method and, for example, a route in which a travel distance of the automated driving vehicle 200 is the shortest may be determined from a geographical point of view for shortening a time required for a round as much as possible by shortening the travel distance of a round doctor DR. The determiner 110 outputs the determined vehicle allocation schedule to the service terminal communicator 120. For example, the vehicle allocation schedule is information including the destinations, the round order, and the expected arrival times shown in FIG. 5.

For example, the service terminal communicator 120 may be a radio communication module for communicating with the communication device 220 of the automated driving vehicle 200 and the like through a network NW. The service terminal communicator 120 transmits the vehicle allocation schedule output by the determiner 110 to the communication device 220 of the automated driving vehicle 200.

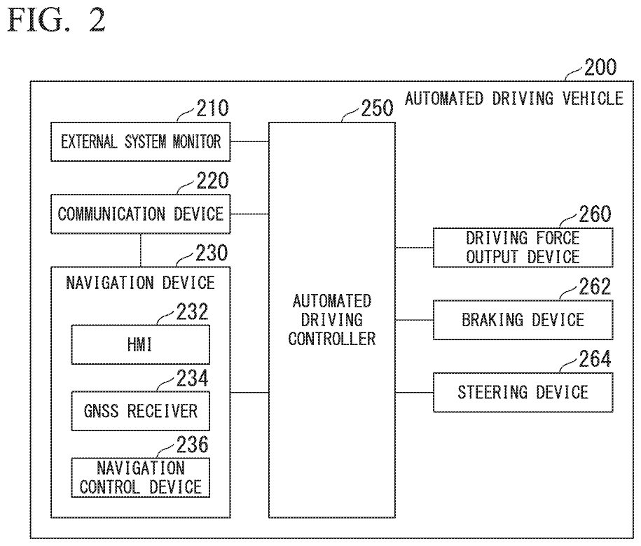

The automated driving vehicle 200, for example, is a vehicle in which one or more users can ride as vehicle occupants and is a vehicle having a function of being able to travel without driving operations being performed. FIG. 2 is a configuration diagram of the automated driving vehicle 200. As shown in FIG. 2, the automated driving vehicle 200, for example, includes an external system monitor 210, a communication device (one example of a second communicator) 220, a navigation device 230, an automated driving controller (one example of an automated driving controller) 250, a driving force output device 260, a braking device 262, and a steering device 264.

The external system monitor 210, for example, includes a camera, a radar, and a light detection and ranging (LIDAR) device, and an object recognizing device performing a sensor fusion process on the basis of outputs thereof, and the like. The external system monitor 210 determines types of objects (particularly, a vehicle, a pedestrian, and a bicycle) present in the vicinity of the automated driving vehicle 200 and outputs the estimated types of the objects to the automated driving controller 250 together with information of locations and speeds thereof.

The communication device 220, for example, is a radio communication module for connection to the network NW or direct connection to another vehicle, a terminal device of a pedestrian, and the like. The communication device 220 performs radio communication on the basis of Wi-Fi, dedicated short range communications (DSRC), Bluetooth (registered trademark), or any other communication specification. A plurality of communication devices 220 according to uses may be provided.

The communication device 220 receives a vehicle allocation schedule transmitted by the service terminal communicator 120 and outputs the received vehicle allocation schedule to the navigation device 230. The navigation device 230 generates route information on the basis of the vehicle allocation schedule output by the communication device 220. The navigation device 230 outputs destinations according to the vehicle allocation schedule output by the communication device 220 and a round order thereof using an HMI (one example of an information output) 232 using speech, an image display, and the like.

For example, the navigation device 230 includes a human machine interface (HMI) 232, a global navigation satellite system (GNSS) receiver 234, and a navigation control device 236. The HMI 232, for example, includes a touch panel-type display device, a speaker, a microphone, and the like. The GNSS receiver 234 performs positioning of the location of the receiver (the location of the automated driving vehicle 200) on the basis of electromagnetic waves arriving from GNSS satellites (for example, GPS satellites). The navigation control device 236, for example, includes a central processing unit (CPU) and various storage devices and controls the overall operation of the navigation device 230.

Map information (a navigation map) is stored in the storage devices. The navigation map is a map that represents roads using nodes and links. The navigation control device 236 determines a route from the location of the automated driving vehicle 200 positioned by the GNSS receiver 234 to a destination designated using the HMI 232 or output from the communication device 220 by referring to the navigation map. An address and the like of the house of the round doctor DR are stored in the storage device. In addition, the navigation control device 236 may transmit the location and the destination of the automated driving vehicle 200 to a navigation server (not shown in the drawing) using the communication device 220 and acquire a route returned from the navigation server. In addition, information of places at which the automated driving vehicle stops for allowing a user to get in or get off and target arrival times may be included in the route. The navigation control device 236 outputs route information of the route determined using one of the methods described above to the communication device 220 and the automated driving controller 250.

The automated driving controller 250 includes one or more processors such as CPUs, MPUs, or the like and various storage devices. The automated driving controller 250 executes automated driving in which the automated driving vehicle 200 is caused to travel automatically along a route based on the route information transmitted by the navigation device 230. The automated driving controller 250, for example, sequentially executes various events. As events, there are a constant-speed traveling event in which the vehicle travels at a constant speed in the same traveling lane, a following traveling event in which the vehicle travels behind a preceding vehicle, a lane changing event, a merging event, a branching event, an emergency stopping event, a tollgate event for passing through a tollgate, a handover event for ending automated driving and switching to manual driving, and the like. In addition, during the execution of such an event, there are also cases in which an action for avoidance is planned on the basis of a surrounding status (the presence of surrounding vehicles and pedestrians, lane narrowing due to roadwork, and the like) of the automated driving vehicle 200.

The automated driving controller 250 generates a target locus along which the automated driving vehicle 200 will travel in the future. The target locus, for example, includes a speed element. For example, the target locus is represented by sequentially aligning points (locus points) at which the automated driving vehicle 200 will arrive. A locus point is a point at which the automated driving vehicle 200 should arrive for every predetermined traveling distance and, in addition thereto, a target speed and a target acceleration for every predetermined sampling time (for example, about a fraction of [sec]) are generated as part of the target locus. In addition, the locus point may be a location at which the automated driving vehicle 200 will arrive at a sampling time for every predetermined sampling time thereof. In such a case, information of a target speed and a target acceleration is represented as an interval between locus points.

The driving force output device 260 outputs a traveling driving force (torque) for causing the vehicle to travel to drive wheels. The driving force output device 260, for example, includes a combination of an internal combustion engine, an electric motor, a transmission gear, and the like and a power ECU controlling these. The power ECU controls the components described above in accordance with information input from the automated driving controller 250 or information input from a driving operator not shown in the drawing.

The braking device 262, for example, includes a brake caliper, a cylinder delivering hydraulic pressure to the brake caliper, an electric motor generating hydraulic pressure in the cylinder, and a brake ECU. The brake ECU performs control of an electric motor in accordance with information input from the automated driving controller 250 or information input from the driving operator such that a brake torque according to a braking operation is output to each vehicle wheel. The braking device 262 may include a mechanism that delivers a hydraulic pressure generated in accordance with an operation on a brake pedal included in the driving operator to the cylinder through a master cylinder as a backup. The braking device 262 is not limited to the configuration described above and may be an electronic control-type hydraulic braking device that delivers a hydraulic pressure of the master cylinder to the cylinder by controlling an actuator in accordance with information input from the automated driving controller 250.

The steering device 264, for example, includes a steering ECU and an electric motor. The electric motor, for example, changes the direction of the steering wheel by applying a force to a rack and pinion mechanism. The steering ECU changes the direction of the steering wheel by driving an electric motor in accordance with information input from the automated driving controller 250 or information input from the driving operator.

Referring back to FIG. 1, the medical examination equipment 300 includes a wearable sensor, a sphygmomanometer, a body measurement device, and the like. The wearable sensor is used for a medical examination involving a blood pressure, a pulse, a body temperature, a respiration rate, an electrocardiogram, an electromyogram, changes (displacements) in the electrocardiogram and the electromyogram, a blood glucose level, a blood oxygen level, a bone density, and the like of a user wearing it. The body measuring device is used for a medical examination of a height, a weight, a body fat percentage, and the like of a user. For measurement of such items, for example, measurement devices dedicatedly used for the items such as an electrocardiograph, an electromyography device, a blood glucose measuring device, a blood oxygen concentration analyzer, a bone density measuring device, and the like may be used. The medical examination equipment 300 includes a data processor that forms results of medical examinations as medical examination result data. The medical examination equipment 300 outputs the medical examination result data formed as data to the medical examination terminal 400.

For example, the medical examination terminal 400 includes an arithmetic operator 410, and a medical diagnosis terminal communicator (one example of a first communicator) 420. For example, the arithmetic operator 410 is realized by a processor such as a central processing unit (CPU) executing a program stored in various storage devices. The arithmetic operator 410 generates biological information on the basis of medical examination result data output by the medical examination equipment 300. The biological information, as shown in FIG. 6, is information in which medical examination result data such as a height, a weight, a blood pressure, a body temperature, a pulse, a respiration rate, and a body fat percentage is added to items such as an ID number, a name, gender, and an age of a user. As such biological information, biological information of each user is generated. The arithmetic operator 410 outputs the generated biological information to the medical diagnosis terminal communicator 420.

For example, the medical diagnosis terminal communicator 420 is a radio communication module for communicating with the service terminal communicator 120 of the service terminal 100, the communication device 220 of the automated driving vehicle 200, and the like through the network NW. The medical diagnosis terminal communicator 420 transmits biological information output by the arithmetic operator 410 to the communication device 220 of the automated driving vehicle 200. The communication device 220 of the automated driving vehicle 200 receives the biological information transmitted by the medical diagnosis terminal communicator 420 and outputs the received biological information to the navigation device 230. The navigation device 230 outputs the biological information output by the communication device 220 using speech, an image display, and the like using the HMI 232.

Next, a process performed by a medical network system according to the first embodiment will be described. The medical network system according to the first embodiment is a system that is used when a round doctor DR riding in the automated driving vehicle 200 makes house calls by visiting user houses inside a round region from a doctor house HHD. On a day on which a diagnosis is performed by the round doctor DR, a user, first, visits a community center CC and receives a medical examination for generating biological information using the medical examination equipment 300. Thereafter, the user returns to his or her house and waits for a visit of the doctor (or waits for a visit of the doctor at the community center CC). Hereinafter, a process performed by the medical network system on the day on which a diagnosis is performed by the round doctor DR will be described.

In the medical examination terminal 400 installed at the community center CC, the arithmetic operator 410 generates biological information on the basis of medical examination result data of the user who has visited for a medical examination. When the medical examination result data of the user is output, the arithmetic operator 410 generates biological information for each user. The arithmetic operator 410 transmits the generated biological information to the service terminal 100 disposed at the service center S through the medical diagnosis terminal communicator 420 and the network NW.

In the service terminal 100 disposed at the service center S, the determiner 110 generates a vehicle allocation schedule. The service terminal 100 transmits a vehicle allocation schedule generated by the determiner 110 and user information stored in various storage devices to the communication device 220 of the automated driving vehicle 200 through the network NW using the service terminal communicator 120. In addition, the service terminal communicator 120 transmits the biological information transmitted by the medical diagnosis terminal communicator 420 of the medical examination terminal 400 to the communication device 220 together with the vehicle allocation schedule and the like. In this way, the medical diagnosis terminal communicator 420 indirectly transmits biological information to the communication device 220 through the service terminal communicator 120.

The communication device 220 outputs the vehicle allocation schedule, the user information, and the biological information transmitted by the service terminal communicator 120 to the navigation device 230. The navigation device 230 outputs various kinds of information corresponding to the user information and the biological information output by the communication device 220 using an image display or the like using the HMI 232.

In addition, in the navigation device 230, the navigation control device 236 generates route information on the basis of destinations and a round order of the destinations according to the vehicle allocation schedule output by the communication device 220. Here, the navigation device 230 generates a route in which the final destination is the doctor house HHD. Thereafter, when the round doctor DR operates the automated driving vehicle 200, a round of a round region using the automated driving vehicle 200 starts from the doctor house HHD.

When the round starts, the navigation device 230 simultaneously outputs a round route and an expected arrival time according to the vehicle allocation schedule output by the communication device 220 using an image display using the HMI 232.

Subsequently, the navigation device 230 outputs biological information of a user having a use ID of “0001” who is a resident of the first round destination (hereinafter, referred to as a “user A”), for example, the A house HA shown in FIG. 3 using an image display using the HMI 232. At this time, in a case in which the biological information of the user A has not yet been transmitted by the medical examination terminal 400 to the communication device 220 through the service terminal 100 and has not been output to the navigation device 230 by the communication device 220, the navigation device 230 does not execute outputting of information corresponding to the biological information of the user A.

The round doctor DR perceives user houses to be rounded for one day by viewing a round route that is displayed as an image in the HMI 232 of the navigation device 230. In addition, the round doctor DR riding in the automated driving vehicle 200 performs a primary diagnosis of a user A while viewing the biological information that is displayed as an image by the HMI 232.

When the automated driving vehicle 200 arrives at the A house HA, and a house call for the user A made by the round doctor DR at the A house HA ends, the automated driving vehicle 200 in which the round doctor DR is riding starts to travel toward the C house HC that is the next visiting place. Before arriving at the C house HC, the navigation device 230 outputs biological information of a user having a use ID of “0003” who is a resident of the C house HC (hereinafter, referred to as a “user C”) as an image display using the HMI 232. The round doctor DR performs a primary diagnosis of the user C while viewing the biological information of the user C.

Thereafter, a visit, a primary diagnosis, and the like similar thereto are repeatedly performed while visiting a house of each user. Then, after visits to all the user houses including the A house HA to the F house HF end, the automated driving vehicle 200 sets the doctor house HHD as a destination. In this way, after ending the last visit, the round doctor DR returns home.

In this way, in the medical network system according to the first embodiment, the medical examination terminal 400 generates biological information including medical examination result data acquired from a result of a medical examination performed by the medical examination equipment 300 disposed at the community center CC. The medical examination terminal 400 transmits the generated biological information to the service terminal 100, and the service terminal 100 transmits the transmitted biological information to the communication device 220 of the automated driving vehicle 200. The communication device 220 outputs the transmitted biological information to the navigation device 230. The navigation device 230 outputs the biological information output by the communication device 220 using speech, an image display, and the like using the HMI 232. For this reason, the round doctor DR riding in the automated driving vehicle 200 and rounding inside the round region can perform a primary diagnosis of users in the automated driving vehicle 200. Accordingly, a state can be formed in which when the round doctor DR arrives at a user house, primary diagnosis of a corresponding user has been completed. As a result, the round doctor DR can eliminate a primary diagnosis time at the user house and thus can shorten the staying time at the user house. As a result, the round doctor DR can efficiently round inside the round region.

### Second Embodiment

Next, a medical network system according to a second embodiment will be described. For example, the medical network system according to the second embodiment includes the service terminal 100, the automated driving vehicle 200, the medical examination equipment 300, and the medical examination terminal 400 shown in FIG. 1. Such a configuration is similar to that of the first embodiment described above, and thus description thereof will be omitted. In addition, in the medical network system according to the second embodiment, mainly a process of generating a vehicle allocation schedule that is performed by a determiner 110 of the service terminal 100, a process of generating a route of an automated driving vehicle 200 that is performed by a navigation device 230, and a process performed by an arithmetic operator 410 of the medical examination terminal 400 are different from those according to the first embodiment. Hereinafter, the medical network system according to the second embodiment will be described by focusing on points different from the medical network system according to the first embodiment.

First, a process of the medical examination terminal 400 will be described. In the medical network system according to the second embodiment, a medical examination of a user who has visited for a medical examination is performed using the medical examination equipment 300. The medical examination terminal 400 generates biological information on the basis of medical examination result data from a result of a medical examination acquired by the medical examination equipment 300 using the arithmetic operator 410. The arithmetic operator 410 generates biological information for each user on the basis of medical examination result data of each user. This point is similar to the first embodiment.

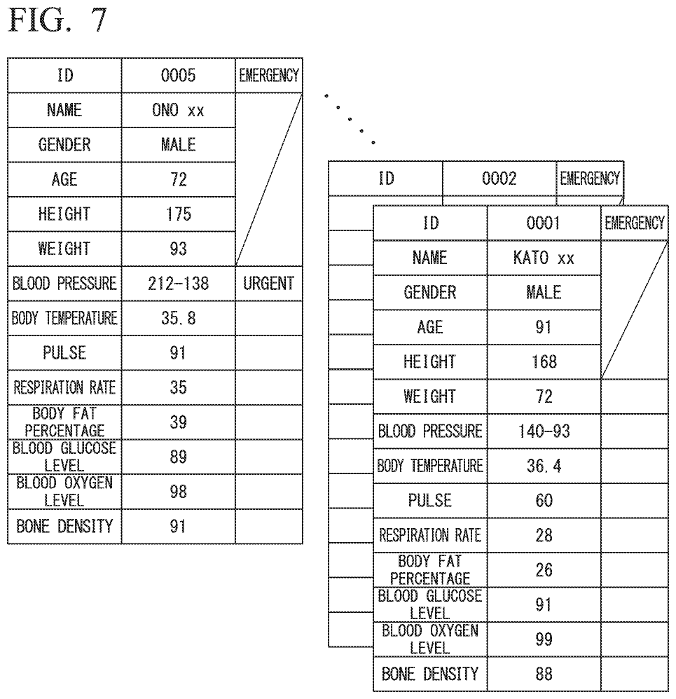

In addition, in the medical network system according to the second embodiment, a second doctor stays at a community center CC. The second doctor performs a primary medical examination of a user on the basis of medical examination result data of the user acquired using the medical examination equipment 300. It may be assumed that the second doctor determines that a secondary medical examination according to a round doctor DR riding in the automated driving vehicle 200 is urgently required as a result of a primary medical examination. In this case, as shown in FIG. 7, the second doctor operates the arithmetic operator 410 of the medical examination terminal 400 such that emergency information indicating “presence” of emergency is added to the biological information. The emergency information is information that can be added to each of items such as “blood pressure”, “body temperature”, “pulse”, “respiration rate”, “body fat percentage”, “blood glucose value”, and “blood oxygen concentration”, and “bone density”. For such items, the second doctor causes the arithmetic operator 410 to add emergency information indicating “presence” of emergency to an item of which a numerical value indicates emergency handling of a user. The arithmetic operator 410 transmits the generated biological information to the service terminal 100 through the network NW using the medical diagnosis terminal communicator 420.

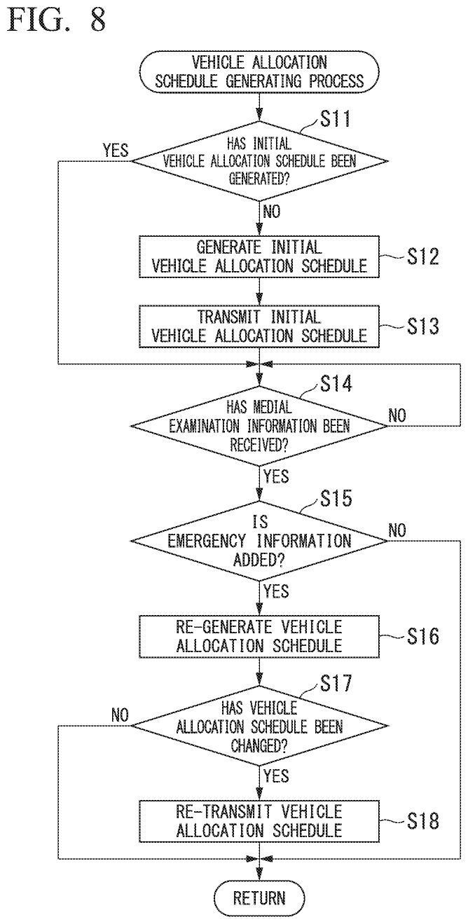

Next, as a process among processes performed by the service terminal 100 that is different from that of the medical network system according to the first embodiment, a vehicle allocation schedule generating process will be described. FIG. 8 is a flowchart showing the sequence of the vehicle allocation schedule generating process. In the vehicle allocation schedule generating process, first, the determiner 110 determines whether or not an initial vehicle allocation schedule has been generated (Step S11).

As a result thereof, in a case in which it is determined that no initial vehicle allocation schedule has been generated, the determiner 110 generates an initial vehicle allocation schedule (Step S12). Subsequently, the determiner 110 transmits the generated initial vehicle allocation schedule to the automated driving vehicle 200 by using the service terminal communicator 120 (Step S13). In addition, in a case in which it is determined that the initial vehicle allocation schedule has been generated in Step S11, the determiner 110 causes the process to immediately proceed to Step S14

Next, the determiner 110 determines whether or not biological information transmitted by the medical diagnosis terminal communicator 420 of the medical examination terminal 400 has been received (Step S14). As a result, in a case in which it is determined that no biological information has been received, the determiner 110 repeats the process of Step S14. In addition, in a case in which no biological information has been received even after a predetermined standby time, for example, 10 minutes, the determiner 110 may immediately determine timeout and end the vehicle allocation schedule generating process.

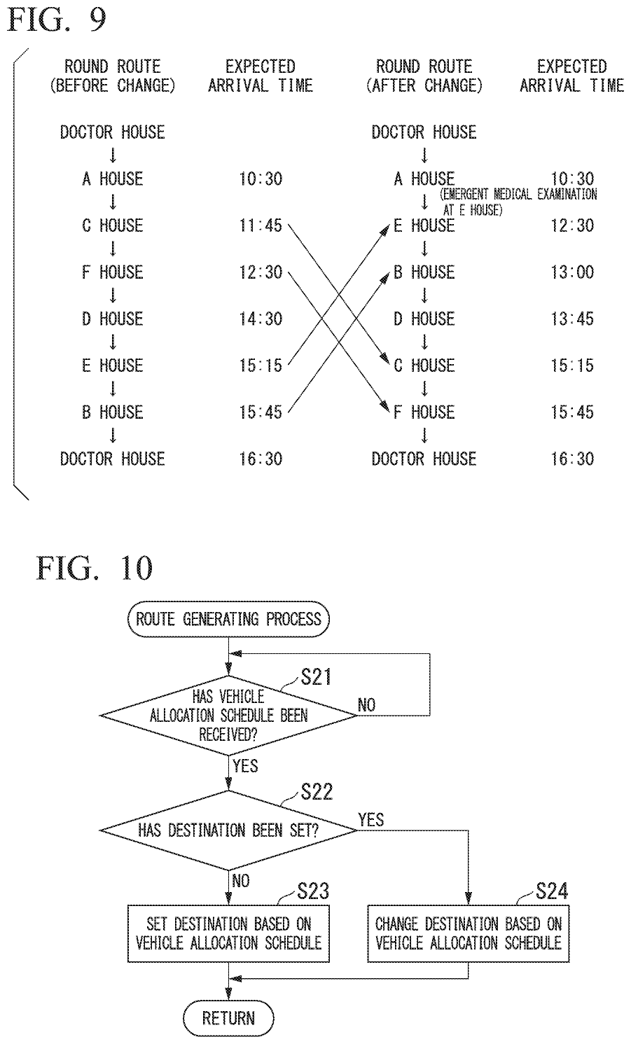

In a case in which it is determined that biological information has been received in Step S14, the determiner 110 determines whether or not emergency information is added to the transmitted biological information (Step S15). As a result thereof, in a case in which emergency information is added to the biological information, the determiner 110 generates a vehicle allocation schedule again on the basis of the emergency information added to the biological information (Step S16). For example, the determiner 110 is assumed to have determined a vehicle allocation schedule of a round route (before change) as shown in FIG. 9, as an initial vehicle allocation schedule in Step S11. The vehicle allocation schedule of the round route (before change) is a vehicle allocation schedule determined on the basis of a geographical point of view and is a vehicle allocation schedule for rounding in order of the A house, the C house, the F house, the D house, the E house, and the B house.

In this state, it is assumed that emergency information having an emergency in the blood pressure of a user having a user ID “0005” is added to the biological information received by the determiner 110 in Step S14 as shown in FIG. 7. Since the user having a user ID “0005” is a resident of the E house, the determiner 110 re-determines a vehicle allocation schedule for advancing a visit to the E house. For this reason, the determiner 110 re-generates a vehicle allocation schedule such that the traveling distance of the automated driving vehicle 200 is the shortest while the visit to the E house is also advanced. For example, the determiner 110 re-generates a vehicle allocation schedule for rounding in order of the A house (visited), the C house, the F house, the D house, the E house, and the B house like a vehicle allocation schedule of the round route (after change) shown in FIG. 9.

Subsequently, the determiner 110 compares the re-generated vehicle allocation schedule with the initial vehicle allocation schedule and determines whether or not the vehicle allocation schedule has been changed (Step S17). As a result, in a case in which it is determined that the vehicle allocation schedule has been changed, the determiner 110 re-transmits the changed vehicle allocation schedule to the automated driving vehicle 200 (Step S18) and ends the vehicle allocation schedule generating process. In addition, in a case in which it is determined that the vehicle allocation schedule has not been changed, the determiner 110 immediately ends the vehicle allocation schedule generating process. In addition, in a case in which it is determined that emergency information is not added in Step S15, re-generation of a vehicle allocation schedule is unnecessary, and the determiner 110 immediately ends the vehicle allocation schedule generating process.

Next, a route generating process will be described. FIG. 10 is a flowchart showing the sequence of a round route generating process. In the route generating process, first, the navigation control device 236 determines whether or not a vehicle allocation schedule has been transmitted by the service terminal communicator 120 of the service terminal 100 (Step S21). As a result thereof, in a case in which it is determined that a vehicle allocation schedule has not been received, the navigation control device 236 repeats the process of Step S21. In addition, in a case in which no biological information has been received even after a predetermined standby time, for example, 10 minutes, the navigation control device 236 may immediately determine timeout and end the route generating process.

In addition, in a case in which it is determined that a vehicle allocation schedule has been received in Step S21, the navigation control device 236 determines whether or not a destination has already been set (Step S22). As a result thereof, in a case in which it is determined that a destination has not been set, the navigation control device 236 sets a destination on the basis of the received vehicle allocation schedule (Step S23) and determines a route of the automated driving vehicle 200. In addition, in a case in which it is determined that a destination has been set, the navigation control device 236 changes the destination corresponding to the transmitted vehicle allocation schedule (Step S24). In this way, the navigation control device 236 ends the route generating process.

In this way, in the medical network system according to the second embodiment, the determiner 110 of the service terminal 100 changes the vehicle allocation schedule on the basis of the emergency information according to a medical examination result transmitted by the medical diagnosis terminal communicator 420. More specifically, the determiner 110 advances a round order of a user house for which emergency information is added. For this reason, the round doctor DR can quickly visit a user house requiring emergence and can perform a round passing through an efficient route. Accordingly, a doctor can efficiently round inside a specific region while handling emergency of a user.

### Third Embodiment

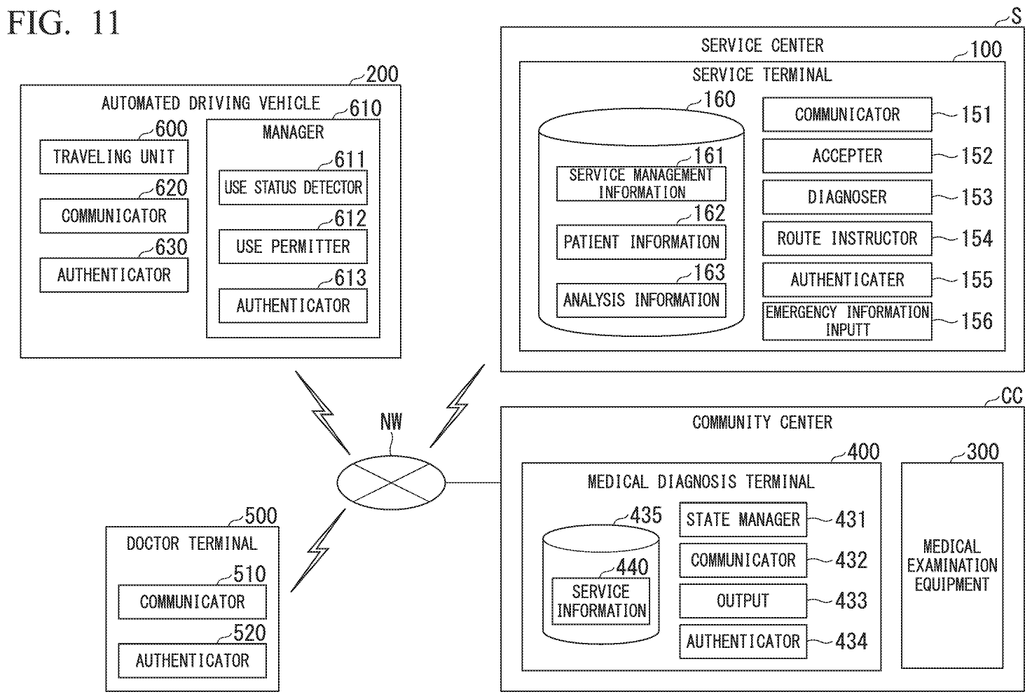

Next, a third embodiment will be described. In the third embodiment, similar to the first embodiment, a medical institution that an automated driving vehicle visits is a hospital H. FIG. 11 is a configuration diagram of a medical network system according to the third embodiment. As shown in FIG. 11, similar to the first embodiment, the medical network system according to the third embodiment includes the service terminal 100, the automated driving vehicle 200, the medical examination equipment 300, and the medical examination terminal 400. In addition, the medical network system according to the third embodiment includes a doctor terminal 500. The doctor terminal 500, for example, is a terminal carried by a doctor riding in the automated driving vehicle 200. Hereinafter, the medical network system according to the third embodiment will be described with focusing on points different from that according to the first embodiment.

As shown in FIG. 11, the service terminal 100 includes a communicator 151, an accepter 152, a diagnoser 153, a route instructor 154, an authenticator 155, an emergency information input 156, and a storage 160. Service management information 161, patient information 162, and analysis information 163 are stored in the storage 160.

The communicator 151 transmits/receives information between the service terminal 100 and the other devices, more specifically, the automated driving vehicle 200, the medical examination terminal 400, and the doctor terminal 500. The accepter 152 accepts various kinds of information transmitted from the automated driving vehicle 200, the medical examination terminal 400, and the doctor terminal 500. The diagnoser 153 performs a diagnosis based on medical examination information transmitted from the doctor terminal 500. In addition, the diagnoser 153 updates information such as results of diagnoses, cases of patients and medical histories, nursing care levels, and the like and stores the updated information in the storage 160.

The route instructor 154 generates a round route of the automated driving vehicle 200 on the basis of the biological information of patients generated by the medical examination terminal 400 and various kinds of information such as visit desire times of patients and the like. In addition, the route instructor 154 determines a doctor responsible for a round on the basis of the round route, plans of a plurality of doctors, and the like. Furthermore, after starting doctor's round, the route instructor 154 updates expected arrival times of round destinations and the updated round route in accordance with location information of the automated driving vehicle 200 in which the doctor is riding, primary diagnosis results transmitted from the doctor terminal 500, and the like.

The authenticator 155 performs authentication for identifying an automated driving vehicle 200 that performs a wound. For example, the authenticator 155 transmits an authentication key of an eye for performing authentication for identifying an automated driving vehicle 200 to the doctor terminal 500. The emergency information input 156 handles a case in which there is a request to deviate from a round according to a normal round route. For example, in a case in which there is a critical patient near the round route, the emergency information input 156 inputs information of the critical patient.

The storage 160 stores a service ID, a vehicle ID, vehicle current location information, a diagnosis status, a diagnosis result attribute, a transit point ID, a transit point task ID, and an expected transit point arrival time as service management information 161. The service ID is information for identifying a doctor, and the vehicle ID is information for identifying an automated driving vehicle 200. The diagnosis status is information relating to a status of a diagnosis performed by a doctor, and the diagnosis result attribute is information relating to a result of a diagnosis performed by a doctor. The transit point ID is information for identifying a transit point that becomes a candidate for a transit point of the automated driving vehicle 200, the transit point task ID is information for identifying a task of arriving at the transit point, and the expected transit point arrival time is information relating to an expected arrival time at the transit point.

Among these, the service ID and the vehicle ID are information that is not updated when the automated driving vehicle 200 rounds, and the vehicle current location information, the diagnosis status, the diagnosis result attribute, the transit point ID, the transit point task ID, and the expected transit point arrival time are information that may be updated when the automated driving vehicle 200 rounds. The storage 160 appropriately provides various kinds of such information for the communicator 151, the diagnoser 153, the route instructor 154, and the authenticator 155.

The storage 160 stores a round target customer ID, a customer contact point, a customer address, a medical history, and a nursing care level as patient information 162. The round target customer ID is information for identifying a target customer who is a customer to become a target when the automated driving vehicle 200 rounds, the customer contact point is a contact point (a telephone number or the like) of the target customer, and the customer address is an address of a residence of the target customer. The medical history is information relating to a medical history of the target customer in the past, and the nursing care level is information relating to a nursing care level of the target customer. Such information is information that is not updated when the automated driving vehicle 200 rounds.

The storage 160 stores a target patient ID, a related diagnosis ID, diagnostic device information, secondary diagnosis information, and the like as analysis information 163. The target patient ID is information for identifying a target patient, and the related diagnosis ID is information relating to a result of a diagnosis relating to the target patient. The diagnostic device information is information relating to a diagnostic device, and the secondary diagnosis information is information relating to a secondary diagnosis result of a target patient. In such information, information other than the secondary diagnosis information is information that is not updated when the automated driving vehicle 200 rounds, and the information other than the secondary diagnosis information is information that may be updated when the automated driving vehicle 200 rounds.

The automated driving vehicle 200 includes a traveler 600, a manager 610, a communicator 620, and an authenticator 630. The manager 610 includes a use status detector 611, a use permitter 612, and an authenticator 613. The use status detector 611 detects a use status of a subject vehicle. The use permitter 612 permits the use of the subject vehicle by referring to an authentication ID or the like. The authenticator 613 authenticates an authentication ID when the subject vehicle is used.

The communicator 620 performs transmission/reception of information between the automated driving vehicle 200 and the other devices such as the service terminal 100, the medical examination terminal 400, and the doctor terminal 500. The authenticator 630 performs authentication for identifying a user (doctor) using the subject vehicle using the service ID and the like that have been transmitted.

In addition, the automated driving vehicle 200 stores vehicle information. Information relating to a vehicle ID, location information (latitude, longitude, and height) of the subject vehicle, compartment use information (use doctor and usable compartment (diagnostic device)), a traveling state, manager information, a service owner ID, a use period, and the like is included in the vehicle information

The medical examination terminal 400 includes a state manager 431, a communicator 432, an output 433, an authenticator 434, and a storage 440. The state manager 431 manages a use state (usable/non-usable and the like) of an adjacent medical examination equipment 300. The communicator 432 performs transmission/reception of information between the automated driving vehicle 200 and the other devices such as the service terminal 100, the automated driving vehicle 200, and the doctor terminal 500.

The output 433 outputs information requested by the state manager and information received by the communicator 432. The authenticator 434 performs authentication for identifying a patient using a target patient ID and the like that have been transmitted. In addition, the medical examination terminal 400 stores a task ID, information of a person responsible for execution, a target patient ID, diagnostic device information, a primary diagnosis result, a task status, a service status, facility location information, and the like as service information.

The doctor terminal 500 includes a communicator 510 and an authenticator 520. The communicator 510 performs transmission/reception of information between the medical examination terminal 400 and the other devices, for example, the service terminal 100, the automated driving vehicle 200, and the doctor terminal 500. The doctor terminal 500 includes a communicator 510 and an authenticator 520.

The communicator 510 performs transmission/reception of information between the doctor terminal 500 and the other devices, more specifically, the service terminal 100, the automated driving vehicle 200, and the medical examination terminal 400. The authenticator 520 performs authentication in the doctor terminal 500. In addition, a patient ID of a target patient, a diagnosis ID, diagnostic device information, route information, and the like are stored in the doctor terminal 500.

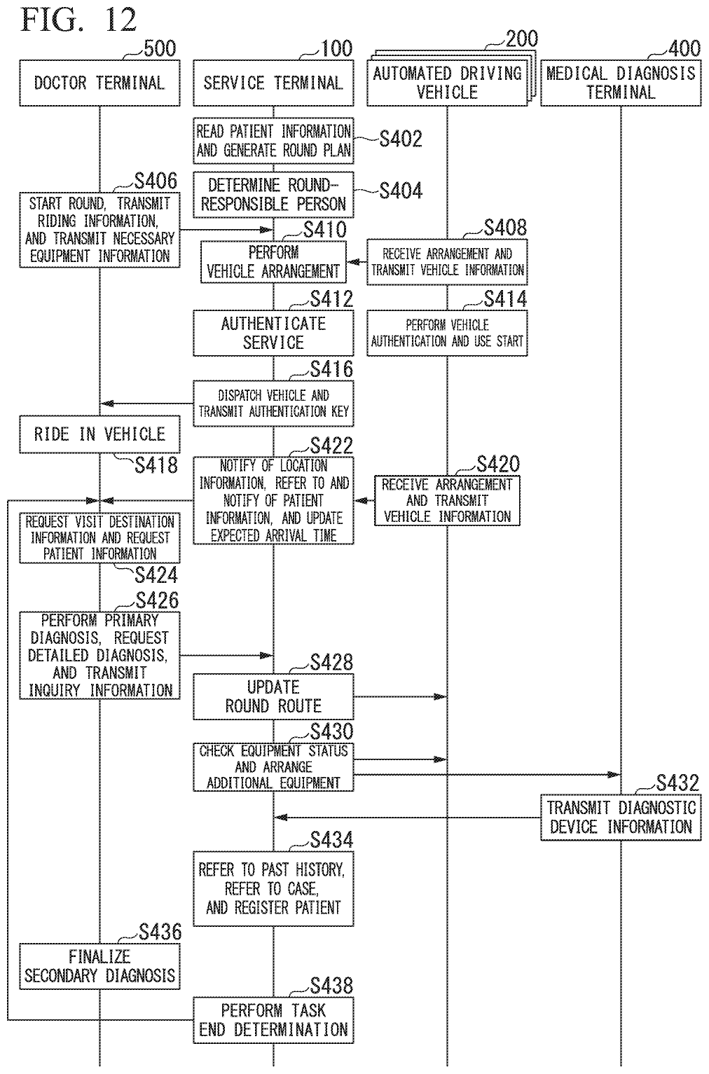

FIG. 12 is a sequence diagram showing a process executed by the medical network system according to the third embodiment. The process executed by the medical network system according to the third embodiment is executed after biological information generated by the medical examination terminal 400 at the community center CC is acquired by the service terminal 100 of the service center S.

As shown in FIG. 12, the service terminal 100 reads the patient information 162 stored in the storage 160 and generates a round plan for the round route based on the patient information of patients (target patients) that become round destinations by using the route instructor 54 (Step S402). Subsequently, the service terminal 100 determines a doctor who becomes a person responsible for rounding (Step S404). For example, a person responsible for rounding may be determined on the basis of an input according to an operator's input operation, or a list of doctors who become candidates for the round, (vacant) schedules of doctors listed in the list, information of a geographical short/long distance from the round route, and the like may be stored in the storage 160, and a person responsible for rounding may be determined on the basis of such conditions. The service terminal 100 transmits a round plan to the doctor terminal 500 of the determined responsible doctor. In addition, when a round plan is transmitted, an arrival time to a customer address and biological information and patient information of a target patient are also transmitted together.

Subsequently, the doctor terminal 500 of the responsible doctor enters start of a round (Step S406). At this time, the responsible doctor transmits riding information according to the round plan and necessary equipment information relating to equipment that is necessary for a medical examination from the doctor terminal 500 to the service terminal 100. The service terminal 100 receives the transmitted riding information and performs arrangement of vehicle allocation. In the arrangement of the automated driving vehicle 200, the service terminal 100 performs arrangement of vehicle allocation of the automated driving vehicle 200 for a vehicle service provider.

The vehicle service provider that has received the arrangement of vehicle allocation transmits vehicle information (a vehicle ID, location information, and the like) of the arranged automated driving vehicle 200 to the service terminal 100 (Step S408). In accordance with reception of vehicle information, the service terminal 100 arranges the vehicle (Step S410). The service terminal 100 performs service authentication for the arranged automated driving vehicle 200 (Step S412).

When service authentication is performed by the service terminal 100, the automated driving vehicle 200 performs vehicle authentication and starts to use the automated driving vehicle 200 (Step S414). The service terminal 100 acquires an authentication key of the automated driving vehicle in which a responsible doctor is riding through service authentication.

The service terminal 100 performs dispatch of a vehicle and transmission of an authentication key (Step S416). The automated driving vehicle 200 performs automated driving and moves toward the place of a responsible doctor through dispatch of the vehicle. In addition, the service terminal 100 transmits (sends) the authentication key of the automated driving vehicle to the doctor terminal 500 (Step S416).

The doctor terminal 500 receives an authentication key transmitted from the service terminal 100. Subsequently, when the automated driving vehicle 200 arrives at the place of the responsible doctor, the responsible doctor rides in the automated driving vehicle 200 with the doctor terminal 500 held (Step S418). When the responsible doctor inputs an authentication key to a predetermined input of the automated driving vehicle 200, the responsible doctor can ride in the automated driving vehicle 200.

In addition, in a case in which necessary equipment needs to be sent by another automated driving vehicle 200 to the automated driving vehicle 200 in which the responsible doctor is riding in accordance with the necessary equipment information transmitted from the doctor terminal 500, the service terminal 100 performs arrangement of vehicle allocation for the vehicle service provider. The vehicle service provider that has received the arrangement of vehicle allocation transmits vehicle information of an automated driving vehicle conveying necessary equipment (hereinafter, referred to as “another automated driving vehicle”) 200 to the service terminal 100 (Step S420).

The service terminal 100 that has received the vehicle information receives the transmitted vehicle information and calculates an arrival location of another automated driving vehicle 200 on the round route on the basis of the received vehicle information. Necessary equipment for the automated driving vehicle 200 is received/sent from/to another automated driving vehicle 200 at this arrival location. The service terminal 100 notifies the doctor terminal 500 of location information of the receiving/sending location (Step S422). In addition, the service terminal 100 updates an expected arrival time at the round destination by referring to the patient information and transmits the updated information to the doctor terminal 500 (Step S422).

In addition, the responsible doctor requests patient information including visiting destination information according to a customer address included in the patient information from the service terminal 100 (Step S424). Subsequently, the responsible doctor performs a primary diagnosis based on the patient information and performs a request for a detailed diagnosis and transmission of inquiry information in accordance with the result of the primary diagnosis (Step S426). Then, the service terminal 100 updates the round route on the basis of the request for a detailed diagnosis and the inquiry information that have been transmitted (Step S428) and transmits the updated round route to the automated driving vehicle 200.

In addition, the service terminal 100 checks the equipment status for the medical examination terminal 400 and performs arrangement of additional necessary equipment on the basis of the result of the primary diagnosis (Step S430). The medical examination terminal 400 transmits diagnostic device information relating to presence/absence of a diagnostic device according to the equipment checking to the service terminal 100 (Step S432).

Then, the service terminal 100 performs patient registration by referring to a history (a medical history) and cases of a patient of the round destination in the past (Step S434). Thereafter, the responsible doctor (the doctor terminal 500) finalizes a secondary diagnosis on the basis of the medical history of the patient in the past, the result of the diagnosis acquired by the diagnostic device, and the like (Step S436). Thereafter, the service terminal 100 performs a task end determination (Step S438) and returns to Step S424 and repeats a similar process when the task has not ended.

In the medical network system according to the third embodiment described above, the service center S (the service terminal 100) determines a round plan and dispatches a doctor, and the doctor moves while performing a diagnosis based on a sensor value acquired by communicating with an in-vehicle diagnostic device. Here, in a case in which a handheld diagnostic device or an in-vehicle diagnostic device is insufficient, additional equipment is conveyed by another automated driving vehicle 200 and is received on the round route, and a diagnosis is performed. Alternatively, additional equipment is dispatched to a relay place (facility). In addition, the diagnosis data is also accumulated on the service center side, a secondary diagnosis is performed on the basis of a numerical analysis, and the doctor finalizes a diagnosis by checking the state.

In this way, in the medical network system according to the third embodiment, a responsible doctor riding in the automated driving vehicle 200 and rounding inside the round region can perform a primary diagnosis of a patient in the automated driving vehicle 200. Accordingly, when arriving at a house of a patient or the like, the responsible doctor can form a state in which a primary diagnosis of the patient is completed. As a result, since the responsible doctor can eliminate a time required for the primary diagnosis at a house such as a house of a patient or the like, a staying time of the patient at the house or the like can be shortened. As a result, the responsible doctor can efficiently round inside the round region.

In addition, by using the medical network system according to the third embodiment, a diagnosis that cannot be easily performed using only the doctor terminal can be executed while moving a facility terminal present at a physical transit point and a service terminal present at a medical facility. For this reason, also in a region in which facilities for diagnoses are scattered at points, diagnoses of various cases similar to those of a large medical facility can be executed at many transit points with high efficiency using a plurality of diagnostic devices and remote service terminals corresponding to a high-level analysis.

As above, while forms for performing the present invention have been described using the embodiments, the present invention is not limited to such embodiments at all, and various modifications and substitutions may be applied within a range not departing from the concept of the present invention. For example, in each of the embodiments described above, although a vehicle allocation schedule is determined by providing the determiner 110 in the service terminal 100 at the service center S, a vehicle allocation schedule may be generated by the automated driving vehicle 200 by providing a determiner in the automated driving vehicle 200. In such a case, for example, the determiner may automatically generate a vehicle allocation schedule at a predetermined time or may generate a vehicle allocation schedule in accordance with an operation of a doctor or the like riding in the automated driving vehicle 200.

In addition, although a doctor stays at the community center CC and performs a primary medical examination in the second embodiment described above, a round doctor DR may perform a primary medical examination on the basis of biological information transmitted by the medical diagnosis terminal communicator 420 to the communication device 220 of the automated driving vehicle 200. Alternatively, the medical diagnosis terminal communicator 420 may transmit biological information to a medical institution such as a hospital, and a primary medical examination may be performed by the medical institution.

In addition, in each of the embodiments described above, although the medical examination terminal 400 indirectly transmits biological information to the communication device 220 of the automated driving vehicle 200 through the service terminal 100, the medical examination terminal 400 may directly transmit biological information to the communication device 220. In a case in which biological information is directly transmitted to the communication device 220, for example, a vehicle allocation schedule may be requested to be generated for the service center S by a round doctor DR, who is riding in the automated driving vehicle 200, performing an operation of communicating with the service center S.

In addition, in each of the embodiments described above, although the determiner 110 of the service terminal 100 determines a vehicle allocation schedule from a point of view of shortening a time required for a round of the round doctor DR as much as possible, the determiner 110 may determine a vehicle allocation schedule from a different point of view. For example, determiner 110 may determine a vehicle allocation schedule for rounding in order of highest to lowest age of users, or a desired house call time of each user may be acquired, and a schedule with desired house call times taken into account may be determined. In addition, the determiner 110 may store a vehicle allocation schedule in advance without calculating the vehicle allocation schedule in detail and output the stored vehicle allocation schedule to the service terminal communicator 120.

In addition, in each of the embodiments described above, although the navigation device 230 simultaneously outputs a round route, an expected arrival time, and biological information of a user using an image display using the HMI 232, those may be alternately displayed as images. In addition, a round route, an expected arrival time, and biological information of users may be displayed as an image in a different form of the navigation device 230. Furthermore, an output form of information output by the HMI 232 may be changeable in accordance with a switching operation or the like. For example, by operating a button corresponding to a speech output, a round route and biological information may be output as speech, and, by operating a button corresponding to display switching, a user whose biological information is output may be switched.

In addition, in each of the embodiments described above, although the medical examination equipment 300 is installed only at the community center CC inside the round region, medical examination equipment 300 may be installed at a plurality of facilities or may be installed at a facility outside the round region. In addition, the medical examination equipment 300 may be installed at facilities other than a specific facility inside/outside the round region and, for example, may be installed at user houses, a doctor house, and the like. Furthermore, the medical examination equipment 300 may be mounted in a vehicle such as the automated driving vehicle 200 or the like.

In addition, in each of the embodiments described above, although a place at which a user is present is fixed to a user house, for example, a current location of a user may be detected using a GPS device or the like, and the automated driving vehicle 200 may be configured to round at the current location of the user. Furthermore, although a departure/arrival location of the automated driving vehicle 200 is a doctor house inside a round region, the departure/arrival location may be a different location. For example, the departure/arrival location may be a doctor house outside the round region or may be a vehicle storage warehouse or the like inside/outside the round region.

In addition, in each of the embodiments described above, although one automated driving vehicle 200 rounds one round region, a plurality of automated driving vehicles 200 may round one round region. A round doctor DR may ride in each of the plurality of automated driving vehicles 200. Alternatively, one or a plurality of automated driving vehicles 200 may round a plurality of round regions.

In addition, in each of the embodiments described above, the determiner 110 may determine a round route in another form in which the automated driving vehicle 200 determines a round route for visiting each user house without depending on results of primary diagnoses. For example, in the second embodiment, a doctor present at the community center CC may determine necessity/non-necessity of visit through a primary medical examination and generate a round route in which visit to a user house to which visit is not necessary is omitted.

### REFERENCE SIGNS LIST

100 service terminal
110 determiner
120 service terminal communicator
200 automated driving vehicle
210 external system monitor
220 communication device
230 navigation device
232 HMI
234 GNSS receiver
236 navigation control device
250 automated driving controller
260 driving force output device
262 braking device
264 steering device
300 medical examination equipment
400 medical diagnosis terminal
410 arithmetic operator
420 medical diagnosis terminal communicator
DR round doctor
NW network
S service center

What is claim is:

1.-8. (canceled)

9. A medical network system comprising:
a medical diagnosis terminal that is installed at a facility at which medical examination equipment is disposed and comprises a first communicator;
an automated driving vehicle in which a doctor rides and comprises a second communicator and an information output; and
a determiner configured to determine a vehicle allocation schedule of the automated driving vehicle to make the automated driving vehicle travel inside a specific region,
a third communicator that is disposed in parallel with the determiner,
wherein the first communicator is configured to directly or indirectly transmit a medical examination result of the medical examination equipment to the second communicator and the third communicator,
wherein the information output is configured to output information based on the medical examination result received by the second communicator to the doctor,
wherein the determiner is configured to change the vehicle allocation schedule on the basis of the medical examination result received by the third communicator, and
wherein the third communicator is configured to transmit the received medical examination result to the second communicator together with the changed vehicle allocation schedule.

10. An external device comprising:
a communicator configured to communicate with a medical diagnosis terminal that is installed at a facility at which medical examination equipment is disposed, a doctor terminal operated by a doctor, and an automated driving vehicle in which the doctor rides; and
a determiner configured to determine a vehicle allocation schedule of the automated driving vehicle to make the automated driving vehicle travel inside a specific region,
wherein the determiner is configured to change the vehicle allocation schedule on the basis of treatment-related information received from the doctor terminal and transmit the determined vehicle allocation schedule to the doctor terminal.

11. The external device according to claim 10, wherein the doctor terminal is configured to transmit riding information relating to arrangement of a vehicle according to the received vehicle allocation schedule to the determiner.

12. The external device according to claim 10, wherein the external terminal is configured to transmit authentication information of the automated driving vehicle to the doctor terminal.

13. The external device according to claim 10,
wherein the determiner is configured to estimate an expected arrival time of the automated driving vehicle at a predetermined location, and transmit the estimated expected arrival time to the doctor terminal.

14. The external device according to claim 10,
wherein the determiner is configured to generate necessary equipment-related information on the basis of the treatment-related information, and transmit the necessary equipment-related information to at least one of the automated driving vehicle and the medical diagnosis terminal.
